(19) United States
(12) Patent Application Publication
(10) Pub. No.: US 2005/0009067 A1
(43) Pub. Date: Jan. 13, 2005

(54) Expression profile of pancreatic cancer

(76) Inventors: Craig Logsdon, Houston, TX (US); Samir Hanash, Ann Arbor, MI (US); Diane Simeone, Ann Arbor, MI (US)

Correspondence Address: MEDLEN & CARROLL, LLP, Suite 350, 101 Howard Street, San Francisco, CA, 94105, US

(21) Appl. No.: 10/848,259

(22) Filed: May 18, 2004

Related U.S. Application Data
(60) Provisional application No. 60/471,656, filed on May 19, 2003.

(51) Int. Cl. C12Q 1/68; G01N 33/53; G01N 33/567

(52) U.S. Cl. 435/6; 435/7.2

(57) ABSTRACT

The present invention relates to compositions and methods for cancer diagnostics, including but not limited to, cancer markers. In particular, the present invention provides gene expression profiles associated with pancreatic cancers. Genes identified as cancer markers using the methods of the present invention find use in the diagnosis and characterization of pancreatic cancer. In addition, the genes provide targets for cancer drug screens and therapeutic applications.

This application claims priority to provisional patent application Ser. No. 60/471,656, filed May 19, 2003, which is herein incorporated by reference in its entirety.

This invention was made in part with government support under Grant No. DK58771 awarded by the National Institute of Diabetes and Digestive Diseases. The Government has certain rights in the invention.

### FIELD OF THE INVENTION

The present invention relates to compositions and methods for cancer diagnostics, including but not limited to, cancer markers. In particular, the present invention provides gene expression profiles associated with pancreatic cancers. The present invention further provides novel markers useful for the diagnosis, characterization, and treatment of pancreatic cancers.

### BACKGROUND OF THE INVENTION

Pancreatic cancer is most frequent adenocarcinoma and has the worst prognosis of all cancers, with a five-year survival rate of <3 percent, accounting for the 4^{th} largest number of cancer deaths in the USA (Jemal et al., CA Cancer J Clin., 53: 5-26, 2003). Pancreatic cancer occurs with a frequency of around 9 patients per 100,000 individuals making it the 11^{th} most common cancer in the USA. Currently the only curative treatment for pancreatic cancer is surgery, but only ˜10-20% of patients are candidates for surgery at the time of presentation, and of this group, only ˜20% of patients who undergo a curative operation are alive after five years (Yeo et al., Ann. Surg., 226: 248-257, 1997; Hawes et al., Am. J. Gastroenterol., 95: 17-31, 2000).

The horrible prognosis and lack of effective treatments for pancreatic cancer arise from several causes. There are currently no effective biomarkers useful for early detection of pancreatic cancer or even to differentiate between pancreatic adenocarcinoma and another major pancreatic disease, chronic pancreatitis. Pancreatic cancer tends to rapidly invade surrounding structures and undergo early metastatic spreading, such that it is the cancer least likely to be confined to its organ of origin at the time of diagnosis (Greenlee et al., 2001. CA Cancer J. Clin., 51: 15-36, 2001). Finally, pancreatic cancer is highly resistant to both chemo- and radiation therapies (Greenlee et al., supra). Currently the molecular basis for these characteristics of pancreatic cancer is unknown. What are needed are improved methods for the early diagnosis and treatment of pancreatic cancer.

### SUMMARY OF THE INVENTION

The present invention relates to compositions and methods for cancer diagnostics, including but not limited to, cancer markers. In particular, the present invention provides gene expression profiles associated with pancreatic cancers. The present invention further provides novel markers useful for the diagnosis, characterization, and treatment of pancreatic cancers.

Accordingly, in some embodiments, the present invention provides a method for characterizing pancreatic tissue in a subject, comprising providing a pancreatic tissue sample from a subject; and detecting the presence or absence of expression of two or more genes (e.g., including, but not limited to, S100P, 14-3-3σ, β4 integrin, CEACAM5, PKM2, CST6, CST4, SERPINB5, FXYD3, BIK, SFN, TRIM29, ITGB4, NT5, IFI27 or S100A6). In some embodiments, the detecting the presence of expression of the two or more genes comprises detecting the presence of mRNA expressed from the two or more genes. For example, in some embodiments, detecting the presence of expression of mRNA expressed from the two or more genes comprises exposing the mRNA to a nucleic acid probe complementary to said mRNA. In other embodiments, detecting the presence of expression of the two or more genes comprises detecting the presence of a polypeptide expressed from the two or more genes. For example, in some embodiments, detecting the presence of a polypeptide expression from the two or more genes comprises exposing the polypeptide to an antibody specific to the polypeptide and detecting the binding of the antibody to the polypeptide. In some embodiments, the subject comprises a human subject. In some embodiments, the sample comprises tumor tissue. In some embodiments, characterizing said pancreatic tissue comprises identifying a stage of pancreatic cancer in the pancreatic tissue. In some embodiments, the method further comprises the step of providing a prognosis to the subject. In some embodiments, the prognosis comprises a risk of developing metastatic pancreatic cancer. In other embodiments, the prognosis comprises a risk of developing pancreatic cancer. In some embodiments, the method further comprises the step of providing a diagnosis to the subject. In some embodiments, the diagnosis comprises a diagnosis of pancreatic cancer. In other embodiments, the diagnosis comprises a diagnosis of chronic pancreatitis.

The present invention additionally comprises a kit for characterizing pancreatic cancer in a subject, comprising a reagent capable of specifically detecting the presence of absence of expression of two or more genes (e.g., including, but not limited to, S100P, 14-3-3σ, β4 integrin, CEACAM5, PKM2, CST6, CST4, SERPINB5, FXYD3, BIK, SFN, TRIM29, ITGB4, NT5, IFI27 or S100A6); and instructions for using the kit for characterizing cancer in the subject. In some embodiments, the reagent comprises a nucleic acid probe complementary to a mRNA expressed from the two or more genes. In other embodiments, the reagent comprises an antibody that specifically binds to a polypeptide encoded by the two or more genes. In certain embodiments, the instructions comprise instructions required by the United States Food and Drug Administration for use in in vitro diagnostic products.

The present invention further provides a method of screening compounds, comprising providing a pancreatic cell sample; and one or more test compounds; and contacting the pancreatic cell sample with the test compound; and detecting a change in expression of two or more genes (e.g., including, but not limited to, S100P, 14-3-3σ, β4 integrin, CEACAM5, PKM2, CST6, CST4, SERPINB5, FXYD3, BIK, SFN, TRIM29, ITGB4, NT5, IFI27 or S100A6) in the pancreatic cell sample in the presence of the test compound relative to the absence of the test compound. In some embodiments, the detecting comprises detecting mRNA expressed by the two or more genes. In other embodiments, the detecting comprises detecting a polypeptide encoded by the two or more genes. In some embodiments, the cell is in vitro. In other embodiments, the cell is in vivo. In some embodiments, the test compound comprises an antisense compound. In certain embodiments, the test compound comprises a drug.

### DESCRIPTION OF THE FIGURES

FIG. 1 shows that pancreatic adenocarcinoma, chronic pancreatitis, and normal pancreas samples can be distinguished on the basis of gene expression profiling. Multiple analyses were conducted for 921 probe-sets. A) PCA analysis. B) Dendrogram indicating the relationship between the samples of pancreatic adenocarcinoma (10), pancreatic cancer cell lines (7), chronic pancreatitis (5), and normal pancreas (5).

FIG. 2 shows the numerical distribution of probe sets differentially expressed in pancreatic adenocarcinoma and chronic pancreatitis. “Lower designates probe sets that were reduced and “higher” designates probe sets that were increased compared with control (FIG. 2A) or chronic pancreatitis (FIG. 2B). “No significant change” indicates that either the differences in expression levels were less than 2-fold or the p values were >0.01.

FIG. 3 shows validation of microarray assessment of mRNA levels using Q-RT-PCR analysis of levels of S100P and 14-3-3σ as representative genes over-expressed in microarray data. Individual microarray data are shown for S100P (A) and 14-3-3σ (B). Samples of tumors (n=5) normal pancreas (n=5) and chronic pancreatitis (CP) (n=5) were prepared and analyzed for S100P and 14-3-3σ using RT-PCR (40 cycles) (C). Quantitative real-time RT-PCR was utilized to quantitate mRNA levels for S100P (D) and 14-3-3σ (E) and data are shown as fold±SE of the level measured in samples of normal pancreas (n=5).

FIG. 4 shows that S100P expressed in NIH3T3 cells transfected with an S100P expression vector stimulates their proliferation. A. Western blot showing the expression of S100P protein after transfection of NIH-3T3 cells with an expression vector bearing s100P. B. S100P expression increased the proliferation of NIH3T3 cells estimated using the MTS assay. C. S100P expression increased the percentage of cells in S-phase.

FIG. 5 shows that S100P protects cells against cell death induced by detachment or 5-FU. A. Cell viability in the presence of S100P. B. Cell viability of wild-type and S100P expressing NIH-3T3 cells cultured in the presence of 5-FU (150 ug/ml).

FIG. 6 shows that S100P expression reduced apoptosis. A. The number of apoptotic cells quantitated for wild-type and S100P expressing cells. B. The number of apoptotic NIH3T3 cells after treatment with 5-FU quantitated. C. Caspase 3 activation as indicated by the reduced levels of the active p20 form in western blots from cell lysates after indicated times of culture on polyHEMA-coated dishes.

FIG. 7 shows that purified S100P stimulated proliferation and survival in NIH3T3 cells. A. Expression and purification of His-S100P protein confirmed by western blot by using a monoclonal S100P antibody. B proliferation dose-response. C. proliferation time-course. D. Survival dose-response. E. Survival time-course.

FIG. 8 shows that purified S100P stimulated Erk activation in NIH3T3 cells. A. S100P activation of Erks was time-dependent. B. S100P activation of Erks was concentration-dependent.

FIG. 9 shows that purified S100P stimulates NF-κB activation in NIH3T3 cells. A. S100P effects on NF-κB were time-dependent. B. S100P activation of NF-κB was also concentration dependent.

FIG. 10 shows that S100P interacts directly with RAGE.

FIG. 11 shows that the effects of S100P on cell proliferation, survival, and signaling were dependent upon RAGE activation. A. S100P stimulation of proliferation was blocked by transfection with a dominant negative RAGE (dnRAGE) or treatment with a RAGE antagonist peptide (AmphP) or anti-RAGE FAB frabments (anti-RAGE) but not by transfection with a full-length RAGE (RAGEFL). B. S100P stimulation of cell survival after treatment with 5-FU was blocked by treatment with a RAGE antagonist peptide (AmphP) or anti-RAGE FAB frabments (anti-RAGE). C. S100P effects on cellular Erk activation was blocked by transfection with a dominant negative RAGE (dnRAGE) construct but not a full-length RAGE (RAGEFL). D. S100P activation of NF-κB was blocked by transfection with a dominant negative RAGE (dnRAGE) construct and treatment with an amphoterin-based peptide antagonist (AmphP), but not by transfection with a full-length RAGE (RAGEFL).

### GENERAL DESCRIPTION

Gene expression profiles provide important information about the molecular characteristics of cancers and can be utilized to distinguish closely related cancer subtypes (Welsh et al., Proc. Natl. Acad. Sci. U.S.A, 98: 1176-1181, 2001; Califano et al., Proc. Int. Conf. Intell. Syst. Mol. Biol., 8: 75-85, 2000). Gene profiling can also be used to develop candidate biomarkers (Rosty et al., Am. J. Pathol., 160: 45-50, 2002) and to identify groups of genes involved in specific functional aspects of tumor biology (Gutgemann et al., Arch. Dermatol. Res., 293: 283-290, 2001). One important consideration in the gene profiling of pancreatic adenocarcinoma is the abundant desmoplastic reaction that occurs in these tumors. These pancreatic tumors are composed of neoplastic cells surrounded by a dense fibrous stroma, which contains proliferating fibroblasts, stellate cells, small endothelial-lined vessels, inflammatory cells, and residual parenchymal components of the pancreas. Therefore, comparisons between adenocarcinomas and normal pancreas fail to account for the contribution of stromal elements, and genes identified by these comparisons are not necessarily specific for pancreatic cancer. Chronic pancreatitis, similar to adenocarcinoma, results in lesions containing abundant stroma that are morphologically identical to that observed in adenocarcinoma. Thus, comparison between adenocarcinomas and chronic pancreatitis allows for the elimination of the stromal contribution as well as for identification of the genes specifically expressed in neoplastic cells of pancreatic tumors (Gress et al., Genes, Chromosomes & Cancer, 19: 97-103, 1997; Gress et al., Oncogene, 13: 1819-1830, 1996; Geng et al., Biotechniques, 25: 434-438, 1998).

Experiments conducted during the course of development of the present invention comprised performing 27 oligonucleotide directed microarray experiments representing 10 pancreatic tumors, 5 samples of chronic pancreatitis, 5 samples of normal pancreas, and 7 pancreatic cancer cell lines. Initial examination of the data using principle component analysis, clustering, and numerical comparisons indicated that pancreatic tumors were more distinct from normal pancreas than from chronic pancreatitis. Expression profiles were then compared between pancreatic adenocarcinoma, pancreatic cancer cell lines, normal pancreas and chronic pancreatitis in order to deduct the stromal contribution and more exactly determine the contribution of neoplastic cells. The selection strategy performed resulted in a list of 158 genes more highly expressed in both pancreatic adenocarcinoma and pancreatic cancer cell lines compared to non-cancerous pancreas.

The selection strategy utilized in this study was designed in part to overcome the obstacle inherent in studies on pancreatic tumors of an abundant desmoplastic reaction. Normal pancreas is composed of primarily acinar cells (˜90%) whereas adenocarcinomas include cancer cells as well as abundant stroma and inflammatory cells. In experiments conducted during the course of development of the present invention, many hundreds of genes were found to be differentially expressed between adenocarcinomas and normal pancreas. Fewer differences were noted between adenocarcinomas and chronic pancreatitis. Chronic pancreatitis, similar to pancreatic cancer, involves a desmoplastic reaction with abundant stroma. Therefore, one explanation for the similarity in expression profiles between tumors and chronic pancreatitis is that many genes expressed in both diseases originate in the stromal components. Previous studies on pancreatic cancer gene expression have not identified the contribution of the stromal elements within pancreatic tumors.

In some embodiments, markers identified during the course of development of the present invention find use as diagnostic markers for the differentiation of pancreatic adenocarcinoma and chronic pancreatitis. For example, four genes, 14-3-3σ (stratifin), S100P, S100A6, and β4 integrin, were selected for further investigation as to their expression in neoplastic components of pancreatic adenocarcinoma. In some embodiments, this molecular profile of pancreatic adenocarcinoma is used to identify genes involved in pancreatic carcinogenesis, identify targets for therapy, elucidate clinical biomarkers, and improve understanding of the molecular basis of pancreatic cancer.

Three of the molecules discovered to be highly specifically expressed in pancreatic adenocarcinoma are members of the S100 protein family, namely S100A6, A11 and P. Immunocytochemistry confirmed the specific localization of S100A6 and P to cells of the neoplastic epithelium. S100P was also observed to be expressed in normal islet cells. The functions of these molecules in pancreatic cancer are currently unknown.

14-3-3σ, also known as stratifin, was also observed to be highly expressed in pancreatic adenocarcinomas. Previously, 14-3-3σ was identified as one of several genes more highly expressed in pancreatic cancer cell lines selected for resistance to chemotherapy than their less resistant counterparts (Sinha et al., Electrophoresis, 20: 2952-2960, 1999). 14-3-3σ was also reported to be among the genes expressed in pancreatic cancer cell lines and bulk tumors but was not previously confirmed as being expressed within neoplastic cells (Ryu et al., Cancer Res., 62: 819-826, 2002).

In experiments conducted during the course of development of the present invention, β4 integrin was highly expressed specifically in neoplastic cells of pancreatic adenocarcinoma. Integrins are dimeric proteins composed of non-covalently associated α and β subunits that mediate cellular adhesion and have been found to be important in the progression and spread of cancer. In normal pancreas, the expression of the fibronectin-binding subunit α5; the laminin-binding subunits α2, α3 and α6; and the vitronectin binding subunit αV have been observed together with the β1, β4 and β5 subunits.

### DEFINITIONS

To facilitate an understanding of the present invention, a number of terms and phrases are defined below:

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass) but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “characterizing pancreatic tissue in a subject” refers to the identification of one or more properties of a pancreatic tissue sample (e.g., including but not limited to, the presence of cancerous tissue, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “cancer marker genes” refers to a gene whose expression level, alone or in combination with other genes, is correlated with cancer or prognosis of cancer. The correlation may relate to either an increased or decreased expression of the gene. For example, the expression of the gene may be indicative of cancer, or lack of expression of the gene may be correlated with poor prognosis in a cancer patient. Cancer marker expression may be characterized using any suitable method, including but not limited to, those described herein.

As used herein, the term “a reagent that specifically detects expression levels” refers to reagents used to detect the expression of one or more genes (e.g., including but not limited to, the cancer markers of the present invention). Examples of suitable reagents include, but are not limited to, nucleic acid probes capable of specifically hybridizing to the gene of interest, PCR primers capable of specifically amplifying the gene of interest, and antibodies capable of specifically binding to proteins expressed by the gene of interest. Other non-limiting examples can be found in the description and examples below.

As used herein, the term “detecting a decreased or increased expression relative to non-cancerous pancreatic control” refers to measuring the level of expression of a gene (e.g., the level of mRNA or protein) relative to the level in a non-cancerous pancreatic control sample. Gene expression can be measured using any suitable method, including but not limited to, those described herein.

As used herein, the term “detecting a change in gene expression (e.g., a change in 14-3-3σ (stratifin), S100P, S100A6, or β4 integrin expression) in said pancreatic cell sample in the presence of said test compound relative to the absence of said test compound” refers to measuring an altered level of expression (e.g., increased or decreased) in the presence of a test compound relative to the absence of the test compound. Gene expression can be measured using any suitable method, including but not limited to, those described in the Examples below.

As used herein, the term “instructions for using said kit for detecting cancer in said subject” includes instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products. The FDA classifies in vitro diagnostics as medical devices and requires that they be approved through the 510(k) or analyte specific reagent (ASR) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use. Where applicable, photographs or engineering drawings should be supplied; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; 8) Any additional information regarding the in vitro diagnostic product requested that is necessary for the FDA to make a substantial equivalency determination. Additional information is available at the Internet web page of the U.S. FDA.

As used herein, the term “pancreatic cancer expression profile map” refers to a presentation of expression levels of genes in a particular type of pancreatic tissue (e.g., chronic pancreatitis, primary, metastatic, and pre-cancerous pancreatic tissues). The map may be presented as a graphical representation (e.g., on paper or on a computer screen), a physical representation (e.g., a gel or array) or a digital representation stored in computer memory. Each map corresponds to a particular type of pancreatic tissue (e.g., chronic pancreatitis, primary, metastatic, and pre-cancerous) and thus provides a template for comparison to a patient sample. In preferred embodiments, maps are generated from pooled samples comprising tissue samples from a plurality of patients with the same type of tissue.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein, the term “initial diagnosis” refers to results of initial cancer diagnosis (e.g. the presence or absence of cancerous cells). An initial diagnosis does not include information about the stage of the cancer or the prognosis.

As used herein, the term “biopsy tissue” refers to a sample of tissue (e.g., pancreatic tissue) that is removed from a subject for the purpose of determining if the sample contains cancerous tissue. In some embodiment, biopsy tissue is obtained because a subject is suspected of having cancer. The biopsy tissue is then examined (e.g., by microscopy) for the presence or absence of cancer.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (Kacian et al., Proc. Natl. Acad. Sci. USA 69:3038 [1972]). Other nucleic acids will not be replicated by this amplification enzyme. Similarly, in the case of T7 RNA polymerase, this amplification enzyme has a stringent specificity for its own promoters (Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target.” In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]), has been modified by several groups to optimize conditions for particular types of cells. The art is well aware of these numerous modifications.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp.16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to compositions and methods for cancer diagnostics, including but not limited to, cancer markers. In particular, the present invention provides gene expression profiles associated with pancreatic cancers. Accordingly, the present invention provides method of characterizing pancreatic tissues, kits for the detection of markers, as well as drug screening and therapeutic applications.

I. Markers for Pancreatic Cancer

The present invention provides markers whose expression is specifically altered in cancerous pancreatic tissues. Such markers find use in the diagnosis and characterization of pancreatic cancer.

A. Identification of Markers

Experiments conducted during the development of the present invention resulted in the identification of genes whose expression level was altered (e.g., increased or decreased) in pancreatic cancer. A series of genes were identified that had altered expression in pancreatic cancer as compared to normal pancreas or chronic pancreatitis (e.g., including, but not limited to, S100P, 14-3-3σ, β4 integrin, CEACAM5, PKM2, CST6, CST4, SERPINB5, FXYD3, BIK, SFN, TRIM29, ITGB4, NT5, IFI27 and S100A6).

B. Detection of Markers

In some embodiments, the present invention provides methods for detection of expression of cancer markers (e.g., pancreatic cancer markers). In preferred embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, mucus, and urine). The present invention further provides panels and kits for the detection of markers. In preferred embodiments, the presence of a cancer marker is used to provide a prognosis to a subject.

The present invention is not limited to the markers described above. Any suitable marker that correlates with cancer or the progression of cancer may be utilized, including but not limited to, those described in the illustrative examples below (e.g., S100P, 14-3-3σ, β4 integrin, CEACAM5, PKM2, CST6, CST4, SERPINB5, FXYD3, BIK, SFN, TRIM29, ITGB4, NT5, IFI27 and S100A6). Additional markers are also contemplated to be within the scope of the present invention (See e.g., Table 2). Any suitable method may be utilized to identify and characterize cancer markers suitable for use in the methods of the present invention, including but not limited to, those described in the illustrative Examples below. For example, in some embodiments, markers identified as being up or down-regulated in pancreatic cancer using the gene expression microarray methods of the present invention are further characterized using tissue microarray, immunohistochemistry, Northern blot analysis, siRNA or antisense RNA inhibition, mutation analysis, investigation of expression with clinical outcome, as well as other methods disclosed herein.

In some embodiments, the present invention provides a panel for the analysis of a plurality of markers. The panel allows for the simultaneous analysis of multiple markers correlating with carcinogenesis and/or metastasis. For example, a panel may include two or more markers identified as correlating with cancerous tissue, metastatic cancer, localized cancer that is likely to metastasize, pre-cancerous tissue that is likely to become cancerous, chronic pancreatitis, and pre-cancerous tissue that is not likely to become cancerous. Depending on the subject, panels may be analyzed alone or in combination in order to provide the best possible diagnosis and prognosis. Any of the markers described herein may be used in combination with each other or with other known or later identified cancer markers.

In other embodiments, the present invention provides an expression profile map comprising expression profiles of cancers of various stages or prognoses (e.g., likelihood of future metastasis). Such maps can be used for comparison with patient samples. Any suitable method may be utilized, including but not limited to, by computer comparison of digitized data. The comparison data is used to provide diagnoses and/or prognoses to patients.

1. Detection of RNA

In some preferred embodiments, detection of pancreatic cancer markers (e.g., including but not limited to, those disclosed herein) is detected by measuring the expression of corresponding mRNA in a tissue sample (e.g., pancreatic tissue). mRNA expression may be measured by any suitable method, including but not limited to, those disclosed below.

In some embodiments, RNA is detected by Northern blot analysis. Northern blot analysis involves the separation of RNA and hybridization of a complementary labeled probe.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to an oligonucleotide probe). A variety of hybridization assays using a variety of technologies for hybridization and detection are available. For example, in some embodiments, the TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

2. Detection of Protein

In other embodiments, gene expression of cancer markers is detected by measuring the expression of the corresponding protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference is utilized.

3. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information providers, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication system). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of metastasis or the presence of cancer or chronic pancreatitis) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

4. Kits

In yet other embodiments, the present invention provides kits for the detection and characterization of pancreatic cancer. In some embodiments, the kits contain antibodies specific for a cancer marker, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

5. In vivo Imaging

In some embodiments, in vivo imaging techniques are used to visualize the expression of cancer markers in an animal (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of the present invention are useful in the diagnosis of cancers that express the cancer markers of the present invention (e.g., pancreatic cancer). In vivo imaging is used to visualize the presence of a marker indicative of the cancer. Such techniques allow for diagnosis without the use of an unpleasant biopsy. The in vivo imaging methods of the present invention are also useful for providing prognoses to cancer patients. For example, the presence of a marker indicative of cancers likely to metastasize can be detected. The in vivo imaging methods of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin Onc 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl) EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m, which does not use chelation with DPTA, is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen-binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

II. Antibodies

The present invention provides isolated antibodies. In preferred embodiments, the present invention provides monoclonal antibodies that specifically bind to an isolated polypeptide comprised of at least five amino acid residues of the cancer markers described herein (e.g., S100P, 14-3-3σ, β4 integrin, CEACAM5, PKM2, CST6, CST4, SERPINB5, FXYD3, BIK, SFN, TRIM29, ITGB4, NT5, IFI27 and S100A6). These antibodies find use in the diagnostic methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a cancer marker of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a cancer marker of the present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

III. Drug Screening

In some embodiments, the present invention provides drug screening assays (e.g., to screen for anticancer drugs). The screening methods of the present invention utilize cancer markers identified using the methods of the present invention (e.g., including but not limited to, S100P, 14-3-3σ, β4 integrin, CEACAM5, PKM2, CST6, CST4, SERPINB5, FXYD3, BIK, SFN, TRIM29, ITGB4, NT5, IFI27 and S100A6). For example, in some embodiments, the present invention provides methods of screening for compounds that alter (e.g., increase or decrease) the expression of cancer marker genes. In some embodiments, candidate compounds are antisense agents (e.g., oligonucleotides) directed against cancer markers. See Section IV below for a discussion of antisense therapy. In other embodiments, candidate compounds are antibodies that specifically bind to a cancer marker of the present invention.

In one screening method, candidate compounds are evaluated for their ability to alter cancer marker expression by contacting a compound with a cell expressing a cancer marker and then assaying for the effect of the candidate compounds on expression. In some embodiments, the effect of candidate compounds on expression of a cancer marker gene is assayed for by detecting the level of cancer marker mRNA expressed by the cell. mRNA expression can be detected by any suitable method. In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to cancer markers of the present invention, have an inhibitory (or stimulatory) effect on, for example, cancer marker expression or cancer marker activity, or have a stimulatory or inhibitory effect on, for example, the expression or activity of a cancer marker substrate. Compounds thus identified can be used to modulate the activity of target gene products (e.g., cancer marker genes) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer.

In one embodiment, the invention provides assays for screening candidate or test compounds that are substrates of a cancer markers protein or polypeptide or a biologically active portion thereof. In another embodiment, the invention provides assays for screening candidate or test compounds that bind to or modulate the activity of a cancer marker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. NatI. Acad. Sci. 87:6378-6382 [1990]; Felici, J. Mol. Biol. 222:301 [1991]).

In one embodiment, an assay is a cell-based assay in which a cell that expresses a cancer marker protein or biologically active portion thereof is contacted with a test compound, and the ability of the test compound to the modulate cancer marker's activity is determined. Determining the ability of the test compound to modulate cancer marker activity can be accomplished by monitoring, for example, changes in enzymatic activity. The cell, for example, can be of mammalian origin.

The ability of the test compound to modulate cancer marker binding to a compound, e.g., a cancer marker substrate, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

Alternatively, the cancer marker is coupled with a radioisotope or enzymatic label to monitor the ability of a test compound to modulate cancer marker binding to a cancer markers substrate in a complex. For example, compounds (e.g., substrates) can be labeled with ¹²⁵I, ³⁵S ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a cancer marker substrate) to interact with a cancer marker with or without the labeling of any of the interactants can be evaluated. For example, a microphysiorneter can be used to detect the interaction of a compound with a cancer marker without the labeling of either the compound or the cancer marker (McConnell et al. Science 257:1906-1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and cancer markers.

In yet another embodiment, a cell-free assay is provided in which a cancer marker protein or biologically active portion thereof is contacted with a test compound and the ability of the test compound to bind to the cancer marker protein or biologically active portion thereof is evaluated. Preferred biologically active portions of the cancer markers proteins to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in 1 5 the assay should be maximal. An FRET binding event can be conveniently measured through standard fluorometric detection means well known in the art (e.g., using a fluorimeter).

In another embodiment, determining the ability of the cancer markers protein to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]). “Surface plasmon resonance” or “BIA” detects biospecific interactions in real time, without labeling any of the interactants (e.g., BlAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize cancer markers, an anti-cancer marker antibody or its target molecule to facilitate separation of complexed from non-complexed forms of one or both of the proteins, as well as to accommodate automation of the assay. Binding of a test compound to a cancer marker protein, or interaction of a cancer marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-cancer marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or cancer marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of cancer markers binding or activity determined using standard techniques. Other techniques for immobilizing either cancer markers protein or a target molecule on matrices include using conjugation of biotin and streptavidin. Biotinylated cancer marker protein or target molecules can be prepared from biotin-NHS (N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with cancer marker protein or target molecules but which do not interfere with binding of the cancer markers protein to its target molecule. Such antibodies can be derivatized to the wells of the plate, and unbound target or cancer markers protein trapped in the wells by antibody conjugation. Methods for detecting such complexes, in addition to those described above for the GST-immobilized complexes, include immunodetection of complexes using antibodies reactive with the cancer marker protein or target molecule, as well as enzyme-linked assays which rely on detecting an enzymatic activity associated with the cancer marker protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit 11:141-8 [1998]; Hageand Tweed J. Chromatogr. Biomed. Sci. Appl. 699:499-525 [1997]). Further, fluorescence energy transfer may also be conveniently utilized, as described herein, to detect binding without further purification of the complex from solution.

The assay can include contacting the cancer markers protein or biologically active portion thereof with a known compound that binds the cancer marker to form an assay mixture, contacting the assay mixture with a test compound, and determining the ability of the test compound to interact with a cancer marker protein, wherein determining the ability of the test compound to interact with a cancer marker protein includes determining the ability of the test compound to preferentially bind to cancer markers or biologically active portion thereof, or to modulate the activity of a target molecule, as compared to the known compound.

To the extent that cancer markers can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, cancer markers protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent W0 94/10300; each of which is herein incorporated by reference), to identify other proteins, that bind to or interact with cancer markers (“cancer marker-binding proteins” or “cancer marker-bp”) and are involved in cancer marker activity. Such cancer marker-bps can be activators or inhibitors of signals by the cancer marker proteins or targets as, for example, downstream elements of a cancer markers-mediated signaling pathway.

Modulators of cancer markers expression can also be identified. For example, a cell or cell free mixture is contacted with a candidate compound and the expression of cancer marker mRNA or protein evaluated relative to the level of expression of cancer marker mRNA or protein in the absence of the candidate compound. When expression of cancer marker mRNA or protein is greater in the presence of the candidate compound than in its absence, the candidate compound is identified as a stimulator of cancer marker mRNA or protein expression. Alternatively, when expression of cancer marker mRNA or protein is less (i.e., statistically significantly less) in the presence of the candidate compound than in its absence, the candidate compound is identified as an inhibitor of cancer marker mRNA or protein expression. The level of cancer markers mRNA or protein expression can be determined by methods described herein for detecting cancer markers mRNA or protein.

A modulating agent can be identified using a cell-based or a cell free assay, and the ability of the agent to modulate the activity of a cancer markers protein can be confirmed in vivo, e.g., in an animal such as an animal model for a disease (e.g., an animal with pancreatic cancer or metastatic pancreatic cancer; or an animal harboring a xenograft of a pancreatic cancer from an animal (e.g., human) or cells from a cancer resulting from metastasis of a pancreatic cancer (e.g., to a lymph node, bone, or liver), or cells from a pancreatic cancer cell line.

This invention further pertains to novel agents identified by the above-described screening assays (See e.g., below description of cancer therapies). Accordingly, it is within the scope of this invention to further use an agent identified as described herein (e.g., a cancer marker modulating agent, an antisense cancer marker nucleic acid molecule, a siRNA molecule, a cancer marker specific antibody, or a cancer marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

IV. Cancer Therapies

In some embodiments, the present invention provides therapies for cancer (e.g., pancreatic cancer). In some embodiments, therapies target cancer markers (e.g., including but not limited to, 14-3-3σ (stratifin), S100P, S100A6, or β4 integrin).

A. Antisense Therapies

In some embodiments, the present invention targets the expression of cancer markers. For example, in some embodiments, the present invention employs compositions comprising oligomeric antisense compounds, particularly oligonucleotides (e.g., those identified in the drug screening methods described above), for use in modulating the function of nucleic acid molecules encoding cancer markers of the present invention, ultimately modulating the amount of cancer marker expressed. This is accomplished by providing antisense compounds that specifically hybridize with one or more nucleic acids encoding cancer markers of the present invention. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of cancer markers of the present invention. In the context of the present invention, “modulation” means either an increase (stimulation) or a decrease (inhibition) in the expression of a gene. For example, expression may be inhibited to potentially prevent tumor proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. in the present invention, the target is a nucleic acid molecule encoding a cancer marker of the present invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in U.S. Patent WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their intemucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl intemucleoside linkages, mixed heteroatom and alkyl or cycloalkyl intemucleoside linkages, or one or more short chain heteroatomic or heterocyclic intemucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the intemucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S—or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisensce oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

B. Genetic Therapies

The present invention contemplates the use of any genetic manipulation for use in modulating the expression of cancer markers of the present invention. Examples of genetic manipulation include, but are not limited to, gene knockout (e.g., removing the cancer marker gene from the chromosome using, for example, recombination), expression of antisense constructs with or without inducible promoters, and the like. Delivery of nucleic acid constructs to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

C. Antibody Therapy

In some embodiments, the present invention provides antibodies that target pancreatic tumors that express a cancer marker of the present invention (e.g., 14-3-3σ (stratifin), S100P, S100A6, or β4 integrin). Any suitable antibody (e.g., monoclonal, polyclonal, or synthetic) may be utilized in the therapeutic methods disclosed herein. In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a cancer marker of the present invention (e.g., 14-3-3σ (stratifin), S100P, S100A6, or β4 integrin), wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeted a cancer marker of the present invention (e.g., 14-3-3σ (stratifin), S100P, S100A6, or β4 integrin). Immunotoxins are conjugates of a specific targeting agent, typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

D. Other Therapeutics

The present invention is not limited to the above-described cancer therapeutics. Additional therapeutics are contemplated including, but not limited to, small molecule therapeutics. For Example in some embodiments, cromolyn (e.g., cromolyn sodium) is used as a small molecule therapeutic. Experiments conducted during the course of development of the present invention indicated that S100P interacts with the RAGE receptor. Previous studies have indicated that cromolyn and other anti-allergic drugs such as olopatadine and amlexanox interact with S100 proteins (Okada et al., Biochem Biophys Res Commun 2002 Apr. 12;292(4):1023-30)The present invention is not limited to a particular mechanism. Indeed, an understanding of the mechanism is not necessary to practice the present invention. Nonetheless is it contemplated that, since cromolyn has been shown to interact with S-100s, that it may also interact with S-100-P, a cancer marker of the present invention, to target cancers by preventing the interaction of S100P with RAGE. Accordingly, in some embodiments, cromolyn is administered alone, or in combination with other therapeutics of the present invention or currently utilized therapeutics, to treat pancreatic cancer.

E. Pharmaceutical Compositions

The present invention further provides pharmaceutical compositions (e.g., comprising the antisense or antibody compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antisense compounds and (b) one or more other chemotherapeutic agents that function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

V. Transgenic Animals Expressing Cancer Marker Genes

The present invention contemplates the generation of transgenic animals comprising an exogenous cancer marker gene of the present invention or mutants and variants thereof (e.g., truncations or single nucleotide polymorphisms). In preferred embodiments, the transgenic animal displays an altered phenotype (e.g., increased or decreased presence of markers) as compared to wild-type animals. Methods for analyzing the presence or absence of such phenotypes include, but are not limited to, those disclosed herein. In some preferred embodiments, the transgenic animals further display an increased or decreased growth of tumors or evidence of cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); and ° C. (degrees Centigrade).

### EXAMPLE 1

Expression Profile of Pancreatic Cancer

This example describes the gene expression profiling of pancreatic cancer and the identification of cancer markers using gene expression profiling.

A. Methods

Pancreatic Tissues and Cell Lines

The primary tumors analyzed in this study were derived from the University of Michigan Health System between 1999 and 2001. Samples of chronic pancreatitis came from both the University of Michigan Health System and the Cooperative Human Tissue Network (CHTN Midwestern Division Columbus, Ohio) and conformed to the policies and practices of the University of Michigan Internal Review Board. Samples of normal pancreas were taken from organ donors provided by the Michigan Transplantation Society (four) or from areas outside regions of pathology in surgically resected pancreata (one). All samples were processed in a similar manner. Frozen samples were embedded in OCT freezing media (Miles Scientific, Naperville, Ill.), cryotome sectioned (5 um) and evaluated by routine hematoxylin and eosin (H&E) stains by a surgical pathologist. Areas of relatively pure tumor, chronic pancreatitis, or normal pancreas were microdissected and these areas were selected for RNA isolation. Pancreatic cancer cell lines BxPC-3, MIA PaCa-2, CFPAC-1, HPAC, MPanc-96, SU.86.86, and SW1990 were obtained from the American Type Culture Collection (Manassas, Va.).

Preparation of cRNA and Gene Chip Hybridization

Total RNA was extracted using Trizol reagent (Invitrogen, Carlsbad, Calif.), followed by clean-up on a RNeasy spin column (Qiagen Inc., Valencia, Calif.) and then used to generate cRNA probes. Preparation of cRNA, hybridization and scanning of the high-density oligonucleotide microarrays [HuGeneFL arrays (7129 probe sets); Affymetrix, Santa Clara, Calif.] were performed according to the manufacturer's protocol (Affymetrix, Santa Clara, Calif.). The preparation of cRNA, hybridization, and scanning of the microarrays were performed according to the manufacturer's protocols, as reported previously (Giordano et al., Am. J Pathol., 159: 1231-1238, 2001).

Data Analysis

Probe intensity values were extracted from the array images using GeneChip 4.0 software (Affymetrix). Each probe set on the HuGeneFL microarray generally consists of 20 coordinated pairs of oligonucleotide features (probes). Within each probe pair, one probe is perfectly complementary (perfect match) whereas the other probe (mismatch) is identical to the complementary probe except for an altered central base. To obtain an expression measure for a given probe set, the mismatch hybridization values were subtracted from the perfect match values, and the average of the middle 50% of these differences was used as the expression measure for that probe set. In this study, 7069 non-control probe sets were analyzed, each of which represents a human transcript. A quantile-normalization procedure was performed to adjust for differences in the probe intensity distribution across different chips. Briefly, a monotone linear spline was applied to each chip that mapped quantiles 0.01 up to 0.99 (in increments of 0.01) exactly to the corresponding quantiles of a tumor chip with low background values as a standard.

Statistical Analysis

For statistical tests, each normalized probe-set expression value, x, was log transformed to log(max(x+100,0)+100), which stabilized the within-group variances between high and low-expression probe-sets. To compare normal, tumor, and chronic pancreatitis samples, a 1-way Analysis of Variance, modeling the log-transformed values was performed for each probe-set as having separate means for each group. Comparison between pairs of groups were performed using the resulting simple contrast tests that are equivalent to ordinary two-sample T-tests except that the variance is estimated using the data from all three groups. Fold changes between groups were calculated of samples by first replacing mean expression values below 100 units by 100 in order to avoid negative values or spuriously large fold changes.

A principle component analysis (PCA) of the log-transformed data was used to provide a visual depiction of the variation in gene expression. The PCA identifies a set of statistically independent projections, or components, of the expression data. The first PC captures the greatest fraction of the overall variance in tumor gene expression compared with any other projection. The second PC captures the greatest fraction of variance subject to being independent of the first projection, and so on. Using any two PCs, a pair of coordinates can be determined for each sample. These coordinates can be used to construct a two-dimensional view that reflects the relative locations of samples in the higher-dimensional space. Samples that fall close together have more similar gene expression values than samples that fall farther apart. For principal component and clustering analysis a set of 921 genes were selected without regard to sample origin by asking that the mean of the tissue samples (cell lines excluded) be larger than 100 units, and the standard deviation divided by the mean be larger than 0.80. For principal component analysis, the data were standardized by subtracting the mean and dividing by the standard deviation for the tissue samples, in order to give each probe-set approximately equal weight.

RT-PCR and Q-RT-PCR

Standard RT-PCR was conducted using total RNA prepared from normal human pancreas, pancreatic adenocarcinomas and samples of chronic pancreatitis, as described above. Reverse transcription was conducted for 45 minutes at 45° C. from 500 ng purified total RNA in a 25 μl volume of Reverse Transcription system reaction mixture by using AMV reverse transcriptase (Promega, Madison, Wis.). Reverse transcription was followed by 35 cycles of standard PCR (1 min denaturation at 94° C., 1 min annealing at 55° C. and 1 min extension at 72° C.). All PCR products were verified by sequencing. Primers designed for human S100P (Genebank accession X65614) were: forward 5′ ATGACGGAACTA GAGACAGCCATGGGC 3′ (SEQ ID NO:1) and reverse, 5′ GGAATCTGTGACAT CTCCAGCGCATCA 3′ (SEQ ID NO:2). Primers designed for human 14-3-3σ (Genebank accession X57348) were: forward 5′ CCGGATCCCTG TGTGTCCCCAGAGCC 3′ (SEQ ID NO:3) and reverse, 5′ CCGAATTCGGCGG TGGCGGGCAACAC 3′ (SEQ ID NO:4). Primers designed for β-actin (Genebank accession BC016045), which was used as a loading control for the RT-PCR reactions, were: forward 5′ ATGATATCGCCGCGCTCGTCGTC 3′ (SEQ ID NO:5) and reverse, 5′ CGCTCGGCCGTGGTGGT GAA 3′ (SEQ ID NO:6). Amplified products were separated on 1.5% agarose gels and visualized by ethidium bromide.

Immunocytochemistry

To identify the cellular source for several of the genes identified in the current study, immunocytochemistry was utilized. For each gene at least three different paraffin-embedded tissue blocks containing adenocarcinoma were labeled. Unstained 4 μm sections were deparaffinized with xylene and rehydrated with ethanol. Antigen retrieval was carried out by repetitive 20 second boiling and cooling cycles for a total of 15 minutes in antigen unmasking solution (Vector Laboratories). Endogenous peroxidase activity was blocked with 6% hydrogen peroxide in methanol and nonspecific binding sites were blocked with normal donkey serum. Primary antibody (14-3-3σ from Santa Cruz Biotechnology, Santa Cruz Calif.; S100P from Research Diagnostics Inc. Flanders N.J.) diluted (14-3-3s 1:250; S100P 1:100) in 2% BSA/0.2% Triton in PBS was added and samples were incubated overnight at 4° C. after which biotinylated secondary antibody (Santa Cruz) was added and incubated for 30 minutes at room temperature followed by Vectastain Elite ABC reagent (Vector Laboratories) and incubation for an additional 30 minutes at room temperature. Finally, slides were developed with DAB substrate (Vector Laboratories), counterstained with hematoxylin, dehydrated with ethanol, fixed with xylene and mounted.

B. Results

Gene Expression Profiles Indicate Relationships between Pancreatic Adenocarcinoma, Normal Pancreas and Chronic Pancreatitis

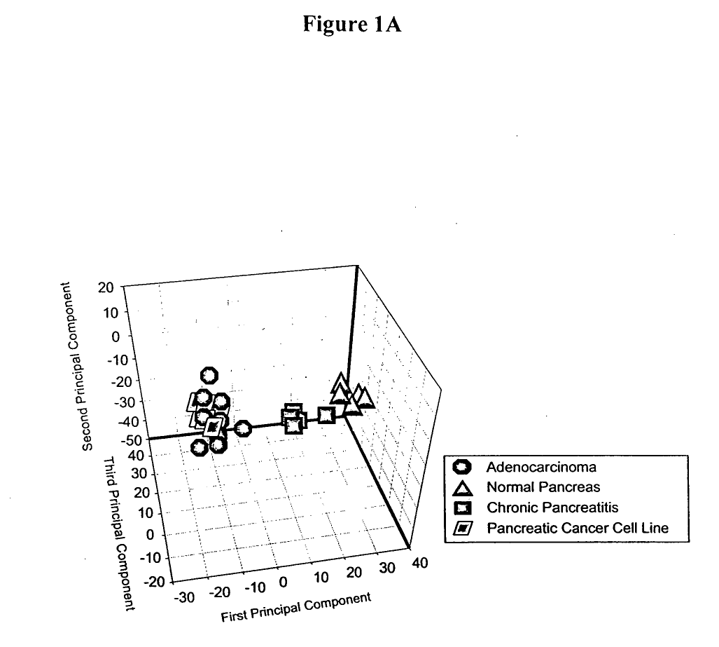

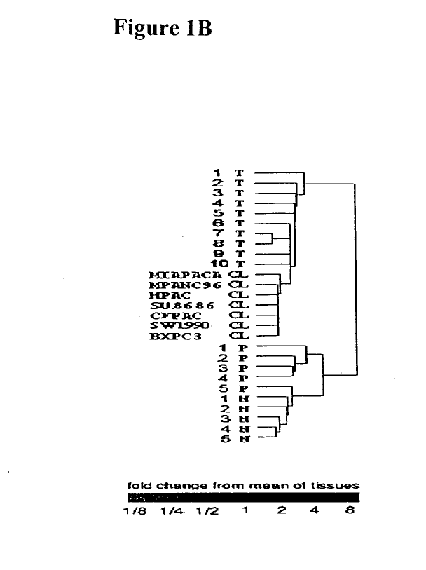

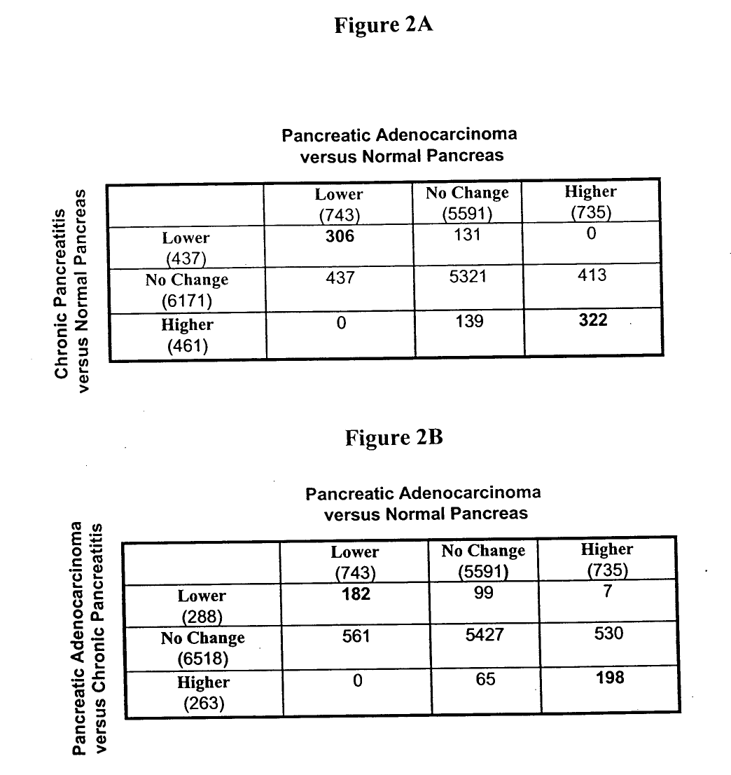

Comprehensive gene expression profiles were generated using high-density oligonucleotide arrays with 7129 probe-sets, which interrogated approximately 6800 genes. To provide a visual assessment of relationships between the 10 adenocarcinoma, 5 chronic pancreatitis, and 5 normal pancreas samples based on gene expression, principal component analysis (PCA) was utilized to locate the three-dimensional view that captured the greatest amount of variability in the data (FIG. 1A). For this analysis, 921 genes were selected on the basis of reasonably high abundance and high sample to sample variability, and the data were standardized to allow each gene to have a nearly equal influence on the outcome. The views generated by PCA indicated substantial differences in gene expression between the three groups of tissue samples (adenocarcinoma, normal, and chronic pancreatitis). A wide margin separated adenocarcinoma samples from normal and slightly less of a margin separated adenocarcinoma from chronic pancreatitis. Chronic pancreatitis was also different than normal as four of five samples clustered together, well separated from the normal samples. When data for 7 pancreatic cancer cell lines was plotted on the same axes, it was observed that the cell lines localized in the vicinity of the adenocarcinoma samples (FIG. 1A).

Further verification of the accuracy of the analysis of relationships between the samples by PCA was obtained using clustering analysis. Eisen matrix formats (Eisen et al., Proc. Natl. Acad. Sci. U.S.A, 95: 14863-14868, 1998) of the 921 genes selected above were utilized to investigate the variation in gene expression, show clusters of coordinately expressed genes, and indicate relationships between specimens. The goals of this analysis were two-fold; first, to see if the mass of data would cluster the samples appropriately; second to allow visualization of the data in order to examine apparent gene patterns and to see if any unexpected patterns were observed. The sample dendrogram revealed the similarities between the experimental samples (FIG. 1B). In this analysis, the adenocarcinoma samples segregated with the pancreatic cell lines, while the normal samples segregated with the chronic pancreatitis samples. Furthermore, four of the five chronic pancreatitis samples clustered together, while one sample clustered with the normal samples. Using a color-coded scheme derived from the TreeView program, a heatmap was created in which the colors are proportional to the fold change from the unweighted average of the adenocarcinoma mean, normal mean, and chronic pancreatitis mean. This analysis showed that groups of genes were differentially expressed in the different samples. For example, a cluster of genes was observed as highly expressed in both pancreatic adenocarcinoma and pancreatic cancer cell lines, but not in normal pancreas or chronic pancreatitis, suggesting that these genes may be expressed specifically in neoplastic epithelium. A different cluster of genes was highly expressed in both adenocarcinoma and chronic pancreatitis samples, but not in either normal pancreas or pancreatic cell lines. Because their expression was not observed in pancreatic cell lines, these genes are likely to originate from stromal elements.

Numerical comparisons between the genes expressed at higher and lower levels in each group of samples were next made. On the basis of ANOVA the comparison between pancreatic adenocarcinoma and normal samples yielded 2313 differences in expression levels at p<0.01, which is many more than the 71 expected on the basis of chance alone. This same comparison between pancreas adenocarcinoma and chronic pancreatitis samples yielded 1086 differences. Using as criterion a fold-change >2.0 and p<0.01, the numbers of genes differentially expressed in adenocarcinoma and chronic pancreatitis were compared to normal pancreas (Table 1). This analysis highlighted the similarities in the distribution of differentially expressed genes in pancreatic adenocarcinoma and chronic pancreatitis. Both diseases shared 322 probe-sets identified as being more highly expressed compared to normal pancreas. Furthermore, ˜70% of the probe-sets that were either more or less highly expressed compared to normal pancreas in chronic pancreatitis were similarly altered in pancreatic adenocarcinoma. Another example of the similarities between the expression profiles of adenocarcinoma and chronic pancreatitis samples is the observation that no gene expressed at higher levels in one was expressed at lower levels in the other, compared to normal pancreas.

TABLE 1
Numeric distribution of probe-sets differentially expressed in
pancreatic adenocarcinoma and chronic pancreatitis indicates
similarities and differences compared to normal pancreas.

| | | T vs N | | |
|---|---|---|---|---|
| | | Lower | No Change | Higher |
| | | (743) | (5591) | (735) |
| CP vs N | Lower | | | |
| T vs CP | (437) | 306 | 131 | 0 |
| | (288) | 182 | 99 | 7 |
| | No Change | | | |
| | (6171) | 437 | 5321 | 413 |
| | (6518) | 561 | 5427 | 530 |
| | Higher | | | |
| | (461) | 0 | 139 | 322 |
| | (263) | 0 | 65 | 198 |

Identification of Genes Differentially Expressed in Pancreatic Adenocarcinoma

In order to identify genes whose expression was specific for the neoplastic epithelium of pancreatic adenocarcinoma, a strategy involving comparisons between profiles for adenocarcinoma, cancer cell lines, normal pancreas and chronic pancreatitis samples was utilized. The initial step for the strategy was a comparison between genes expressed in pancreatic adenocarcinoma versus chronic pancreatitis and versus normal pancreas (Table 1). This comparison highlights features unique to adenocarcinoma and indicates the existence of 198 probe-sets, representing 188 genes, whose expression levels were higher versus both normal and chronic pancreatitis at the 2-fold level (and p<0.01 in both comparisons). In order to further differentiate between genes arising in neoplastic epithelium and those arising in stroma, a comparison of the levels of expression of these 188 genes in pancreatic cancer cell lines to normal pancreas was performed and genes were accepted whose mean expression levels in the cancer cell lines was at least 2-fold higher than in normal pancreas. This comparison resulted in a 16% reduction in the number of selected genes to a final list of 158 genes. The list was restricted to genes expressed at greater than 3-fold in adenocarcinoma compared to both normal and chronic pancreatitis, and also in cancer cell lines compared to normal. Exemplary genes are shown in Table 2. The Table also includes the fold-increases observed in the means of the adenocarcinoma samples compared to normal pancreas and chronic pancreatitis samples, as well as for the pancreatic cancer cell lines compared to normal pancreas. Genes in the list were categorized on the basis of functional data derived from several sources provided by the NCBI including the Mendelian Inheritance in Man (OMIM) site, the Cancer Genome Anatomy Project (CGAP) and Pubmed.

TABLE 2

| Affy | Gene | | | | |
|---|---|---|---|---|---|
| probe_set | Name | Unigene Comment | T/N | T/P | C/N |
| X89986_s_at | BIK | BCL2-interacting killer (apoptosis-inducing) | 20 | 20 | 15 |
| M31516_s_at | DAF | decay accelerating factor for complement (CD55, Cromer | 4 | 3 | 9 |
| | | blood group system) | | | |
| X57348_s_at | SFN | Stratifin (14-3-3σ) | 24 | 8 | 26 |
| L24203_at | TRIM29 | tripartite motif-containing 29 (ATDC) | 20 | 29 | 15 |
| AF001294_at | TSSC3 | tumor suppressing subtransferable candidate 3 | 12 | 5 | 18 |
| J04093_s_at | UGT1A6 | UDP glycosyltransferase 1 family, polypeptide A6 | 7 | 7 | 20 |
| X16662_at | ANXA8 | annexin A8 | 7 | 7 | 24 |
| U17077_at | BENE | BENE protein | 5 | 3 | 5 |
| X63629_at | CDH3 | cadherin 3, type 1, P-cadherin (placental) | 9 | 7 | 6 |
| HG2797- | CLTB | clathrin, light polypeptide (Lcb) | 20 | 7 | 11 |
| HT2906_s_at | | | | | |
| M28249_at | ITGA2 | integrin, alpha 2 (CD49B, alpha 2 subunit of VLA-2 | 7 | 4 | 15 |
| | | receptor) | | | |
| M59911_at | ITGA3 | integrin, alpha 3 (antigen CD49C, alpha 3 subunit of VLA-3 | 3 | 4 | 6 |
| | | receptor) | | | |
| X53587_at | ITGB4 | integrin, beta 4 | 34 | 11 | 93 |
| S72493_s_at | KRT16 | keratin 16 (focal non-epidermolytic palmoplantar | 25 | 11 | 8 |
| | | keratoderma) | | | |
| Z19574_rna1_{—} | KRT17 | keratin 17 | 97 | 22 | 57 |
| at | | | | | |
| Y00503_at | KRT19 | keratin 19 | 26 | 8 | 15 |
| M13955_at | KRT7 | keratin 7 | 8 | 3 | 11 |
| U42408_at | LAD1 | ladinin 1 | 38 | 5 | 38 |
| U03057_at | SNL | singed (Drosophila)-like (sea urchin fascin homolog like) | 11 | 3 | 14 |
| M12125_at | TPM2 | tropomyosin 2 (beta) | 9 | 3 | 4 |
| HG2259- | TUBA1 | tubulin, alpha 1 (testis specific) | 12 | 7 | 7 |
| HT2348_s_at | | | | | |
| X62515_s_at | HSPG2 | heparan sulfate proteoglycan 2 (perlecan) | 9 | 3 | 5 |
| L34155_at | LAMA3 | laminin, alpha 3 (nicein (150 kD), kalinin (165 kD), BM600 | 10 | 9 | 13 |
| | | (150 kD), epilegrin) | | | |
| U17760_rna1_{—} | LAMB3 | laminin, beta 3 (nicein (125 kD), kalinin (140 kD), BM600 | 27 | 9 | 52 |
| at | | (125 kD)) | | | |
| U31201_cds2_{—} | LAMC2 | laminin, gamma 2 (nicein (100 kD), kalinin (105 kD), BM600 | 15 | 8 | 18 |
| s_at | | (100 kD), Herlitz junctional epidermolysis bullosa)) | | | |
| X55740_at | NT5 | 5′ nucleotidase (CD73) | 9 | 9 | 6 |
| U53830_at | IRF7 | interferon regulatory factor 7 | 6 | 3 | 6 |
| X67325_at | IFI27 | interferon, alpha-inducible protein 27 | 48 | 9 | 29 |
| U66711_rna1_{—} | LY6E | lymphocyte antigen 6 complex, locus E | 3 | 3 | 5 |
| s_at | | | | | |
| X70040_at | MST1R | macrophage stimulating 1 receptor (c-met-related tyrosine | 9 | 7 | 9 |
| | | kinase) | | | |
| U64197_at | SCYA20 | small inducible cytokine subfamily A (Cys-Cys), member 20 | 3 | 4 | 6 |
| X12447_at | ALDOA | aldolase A, fructose-bisphosphate | 5 | 3 | 4 |
| X01630_at | ASS | argininosuccinate synthetase | 11 | 3 | 8 |
| J04469_at | CKMT1 | creatine kinase, mitochondrial 1 (ubiquitous) | 12 | 9 | 10 |
| U91316_at | HBACH | cytosolic acyl coenzyme A thioester hydrolase | 4 | 4 | 6 |
| L41668_rna1_{—} | GALE | galactose-4-epimerase, UDP- | 6 | 4 | 9 |
| at | | | | | |
| J03934_s_at | NQO1 | NAD(P)H dehydrogenase, quinone 1 | 10 | 6 | 12 |
| D25328_at | PFKP | phosphofructokinase, platelet | 17 | 3 | 35 |
| U18919_at | NBP | nucleotide binding protein | 6 | 6 | 13 |
| HG3033- | SF3A2 | splicing factor 3a, subunit 2, 66 kD | 6 | 6 | 9 |
| HT3194_at | | | | | |
| HG2465- | TFAP2A | transcription factor AP-2 alpha (activating enhancer-binding | 3 | 4 | 10 |
| HT4871_at | | protein 2 alpha) | | | |
| L17131_rna1_{—} | UBE2M | ubiquitin-conjugating enzyme E2M (homologous to yeast | 5 | 4 | 25 |
| at | | UBC12) | | | |
| L06147_at | GOLGA2 | golgi autoantigen, golgin subfamily a, 2 | 4 | 4 | 4 |
| X92814_at | HREV107 | similar to rat HREV107 | 3 | 3 | 5 |
| M91670_at | E2-EPF | ubiquitin carrier protein | 4 | 4 | 4 |
| X60673_rna1_{—} | AK3 | adenylate kinase 3 | 5 | 5 | 3 |
| at | | | | | |
| X54941_at | CKS1 | CDC28 protein kinase 1 | 6 | 3 | 10 |
| M91083_at | C11orf13 | chromosome 11 open reading frame 13 | 5 | 3 | 4 |
| L35240_at | ENIGMA | enigma (LIM domain protein) | 6 | 3 | 7 |
| L36645_at | EPHA4 | EphA4 | 4 | 4 | 8 |
| M63904_at | GNA15 | guanine nucleotide binding protein (G protein), alpha 15 | 13 | 3 | 6 |
| | | (Gq class) | | | |
| X57579_s_at | INHBA | inhibin, beta A (activin A, activin AB alpha polypeptide) | 20 | 4 | 12 |
| U01062_at | ITPR3 | inositol 1,4,5-triphosphate receptor, type 3 | 6 | 4 | 8 |
| M35878_at | IGFBP3 | insulin-like growth factor binding protein 3 | 28 | 3 | 22 |
| X76029_at | NMU | neuromedin U | 4 | 4 | 9 |
| L40904_at | PPARG | peroxisome proliferative activated receptor, gamma | 7 | 6 | 12 |
| U33053_at | PRKCL1 | protein kinase C-like 1 | 5 | 4 | 5 |
| D38583_at | S100A11 | S100 calcium-binding protein A11 (calgizzarin) | 16 | 3 | 17 |
| HG2788- | S100A6 | S100 calcium-binding protein A6 (calcyclin) | 44 | 4 | 38 |
| HT2896_at | | | | | |
| X65614_at | S100P | S100 calcium-binding protein P | 22 | 17 | 17 |
| X75342_at | SHB | SHB adaptor protein (a Src homology 2 protein) | 3 | 3 | 4 |
| M97936_at | STAT1 | signal transducer and activator of transcription 1, 91 kD | 12 | 3 | 9 |
| L40379_at | TRIP10 | thyroid hormone receptor interactor 10 | 17 | 4 | 19 |
| V00574_s_at | HRAS | v-Ha-ras Harvey rat sarcoma viral oncogene homolog | 6 | 5 | 10 |
| X16354_at | CEACAM1 | carcinoembryonic antigen-related cell adhesion molecule 1 | 13 | 7 | 14 |
| | | (biliary glycoprotein) | | | |
| M29540_at | CEACAM5 | carcinoembryonic antigen-related cell adhesion molecule 5 | 59 | 59 | 46 |
| M18728_at | CEACAM6 | carcinoembryonic antigen-related cell adhesion molecule 6 | 182 | 4 | 91 |
| | | (non-specific cross reacting antigen) | | | |
| L13210_at | LGALS3 | lectin, galactoside-binding, soluble, 3 binding protein | 103 | 4 | 103 |
| | BP | | | | |
| U40434_at | MSLN | Mesothelin | 7 | 3 | 7 |
| X56494_at | PKM2 | pyruvate kinase, muscle | 18 | 10 | 43 |
| D26579_at | ADAM8 | a disintegrin and metalloproteinase domain 8 | 4 | 4 | 5 |
| U62800_at | CST6 | cystatin E/M | 20 | 20 | 24 |
| X54667_at | CST4 | cystatin S | 39 | 13 | 13 |
| U09937_rna1_{—} | PLAUR | plasminogen activator, urokinase receptor | 11 | 5 | 5 |
| s_at | | | | | |
| U04313_at | SERPIN | serine (or cysteine) proteinase inhibitor, clade B | 24 | 20 | 25 |
| | B5 | (ovalbumin), member 5 | | | |
| X93036_at | FXYD3 | FXYD domain-containing ion transport regulator 3 | 71 | 23 | 68 |
| Y07604_at | NME4 | non-metastatic cells 4, protein expressed in | 11 | 4 | 17 |
| U33632_at | KCNK1 | potassium channel, subfamily K, member 1 (TWIK-1) | 7 | 5 | 5 |
| K03195_at | SLC2A1 | solute carrier family 2 (facilitated glucose transporter), | 9 | 4 | 18 |
| | | member 1 | | | |
| X57522_at | TAP1 | transporter 1, ATP-binding cassette, sub-family B | 9 | 4 | 8 |
| | | (MDR/TAP) | | | |

Validation of Microarray Data and Neoplastic Epithelial Cell Gene Expression Identification Strategy

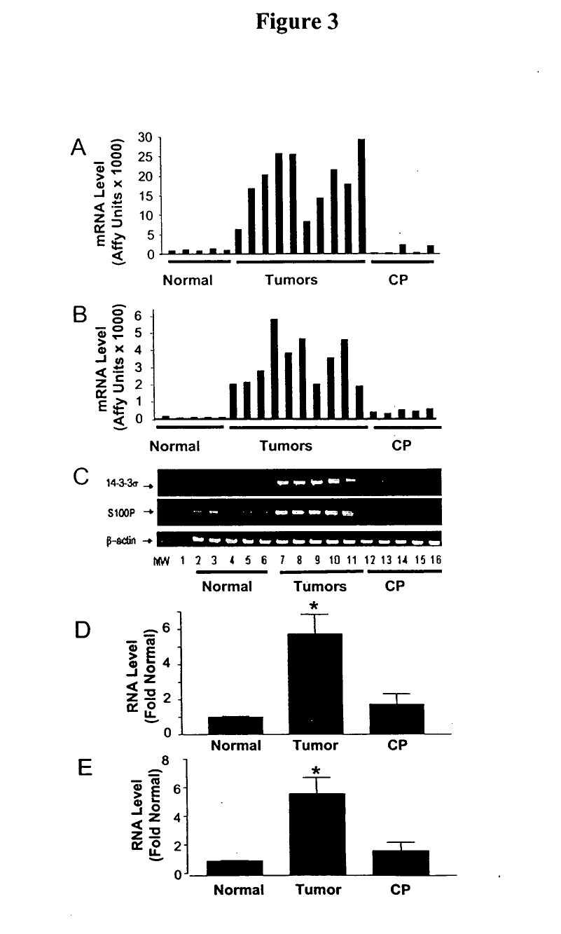

As a means of validating that the microarray data accurately reflect mRNA levels, RT-PCR and quantitative RT-PCR were used to independently examine mRNA levels for two representative genes, S100P and 14-3-3σ, in five separate samples each of normal pancreas, pancreatic adenocarcinoma, and chronic pancreatitis. Affymetrix data for SLOOP (FIG. 3A) and 14-3-3s (FIG. 3B) indicated that they were highly expressed in all 10 adenocarcinomas but none of the 10 non-tumor samples. RT-PCR using high numbers of cycles showed strong bands only in pancreatic tumors (FIG. 3C). In comparison, a weak band was noticed for S100P in some of the normal samples and 14-3-3σ was not present in non-tumor samples. Quantitative PCR verified the significant difference between mRNA expression levels in tumor versus non-tumor samples (FIG. 3D).

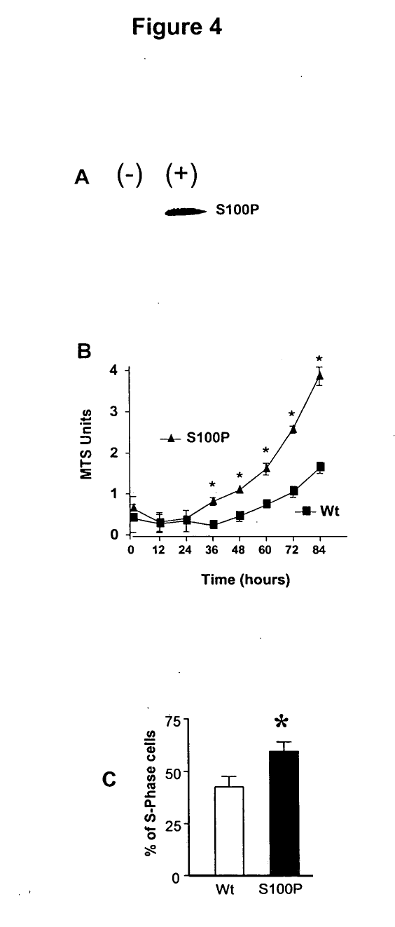

To validate the neoplastic epithelial gene expression identification strategy, the cell type in which four genes, S100P, 14-3-3σ, β4 integrin and S100A6 are expressed was determined in tumors using immunocytochemistry. Expression of these four genes was localized to neoplastic epithelial cells within the tumors (FIG. 4B,E,H,K). None of the genes were observed to be expressed in stromal cells in chronic pancreatitis (FIG. 4C,F,I,L). Likewise, these genes were not expressed in normal acinar or duct cells (FIG. 4A,D,G,J). S100P was expressed to some extent in normal pancreatic islets, which explains the faint bands observed in the RT-PCR analysis. These results support the validity of the microarray selection criteria utilized in the current study.

To further understand the generality of the discovery of these molecules in pancreatic adenocarcinoma, 14-3-3σ, S100P, S100A6 and β4 integrin immunolocalization in paraffin embedded samples from 28 human adenocarcinoma tumors was examined. Each of these molecules was expressed within the neoplastic epithelial cells of all 28 samples (100%). These results identify 14-3-3σ, S100P, S100A6 and β4 integrin as potential histological biomarkers for pancreatic adenocarcinoma.

### EXAMPLE 2

Characterization of S100P

This example describes further characterization of the S100P cancer marker of some embodiments of the present invention.

A. Methods

Development of Stable Cell Lines

NIH3T3 cells transfected using lipofectamine reagent (Invitrogen, Carlsbad, Calif.) with plasmids encoding either a full-length S100P cDNA or a dominant negative RAGE cloned into pcDNA3.1 vector and selected for resistance to G418 (0.5 mg/ml). Wild-type and stably transfected NIH3T3 cells were routinely cultured in DMEM with 10% FBS at 37° C. in a humidified atmosphere of 5% CO₂.

SDS-PAGE, Western Blot Analysis, and Co-immunoprecipitation

Western blot analysis was utilized for the detection of S100P, RAGE, as well as activated Erks and caspase 3 by minor modifications of previously published methods. Cell lysates were prepared and separated by SDS polyacrylimide gel electrophoresis and transferred to nitrocellulose. Membranes were blocked by overnight incubation at 4° C. in 5% milk solution. S100P was detected usingmonoclonal antibodies (Transduction Laboratories, San Diego, Calif.) by incubating the membrane overnight at 4° C. in antibody diluted 1:100 in 5% milk solution. RAGE was detected using goat polyclonal antibodies (Santa Cruz, Santa Cruz, Calif.) by incubating the transferred membrane in for one hour at room temperature with antibody diluted 1:100 in 5% milk solution. Caspase 3 activation was estimated by detection of pro-caspase 3 and active caspase 3 fragments using rabbit polyclonal antibodies against full length caspase 3 (Santacruz Biotech, Calif.) by incubating the membrane at 4° C. for overnight with antibody diluted 1:100 in 5% milk solution. Erk activation was estimated by detection of phosphorylated forms of Erk 1 and 2 using phospho-p44/42 MAP kinase (Thr202/Tyr204) antibody (Cell Signaling, Beverly, Mass.) and, as a loading control after striping, a rabbit polyclonal antibody for total Erk 1 & 2 (Santa Cruz, Santa Cruz, Calif.) by incubating the membrane at 4° C. overnight with antibody diluted 1:100 in 5% milk solution. Second antibody anti-mouse, anti-rabbit, or anti-goat IgG+HRP was incubated for one hour at room temperature and the signal was detected by ECL detection system (Amersham) as per manufacturer protocol.

For co-immunoprecipitation experiments, cell lysates were incubated in the absence or presence of S100P (1 μg) at 4° C. overnight. S100P was immunoprecipitated using mouse monoclonal antibody against S100P (Transduction Laboratories, San Diego, Calif.), for 6 hours at 4° C. and IgG immobolized beads (Pierce, Rockford, Ill.). Antibody associated proteins were electrophoresed on 10% polyacrylamide gel and transferred to nitrocellulose membrane. Transferred membrane was blocked by 5% milk solution overnight at 4° C. RAGE was detected as described.

Cell Growth studies

Cell growth was analyzed by using MTS reagent (Promega, Madison, Wis.) as per manufactures directions. For studies on the effects of S100P expression, vector alone and three different clonal S100P stably transfected cell lines (1000 cells/well) were seeded into 96 well plates and cell growth was studied from 0-120 hrs. For studies on the effects of exogenously applied S100P, purified S100P was added at indicated concentrations for specific times. MTS was added to the wells one hour before taking the photometric reading.

Expression and Purification of Bacterial S100P

Full length S100P cDNA was cloned into pTrcHis2 vector (Invitrogen, Carlsbad, Calif.) and transformed into one shot TOP10 competent E.coli. The bacterial culture was incubated at 37° C. to an OD₆₀₀=0.6, then IPTG (1 mM) was added and the bacteria were cultured for another 3 hrs. His-S100P was purified using Probond resin column as described by the manufacturer (Invitrogen, Carlsbad, Calif.). The fraction was further dialyzed against 10 mM Tris, pH 8.0, containing 01% Triton X 100 overnight at 4° C. using a Slide-A-Lyzer 10K (Pierce, Rockford, Ill.). Dialyzed protein was further concentrated by Centricon centrifugal filter device YM10 (Millipore, Bedford, Mass.). The purified S100P protein was confirmed by western blot and ELISA and used for in vitro experiments.

Induction of Apoptosis

Apoptosis was induced in NIH3T3 cells either by prevention of cell adhesion to a solid substrate (anoikis) or by use of 5-flurouracil. To prevent cell adhesion, 6 well plates were covered (3 ml/well) with a solution of polyhydroxyethylmethacrylate (polyHEMA; Sigma-Aldrich) dissolved at 10 mg/ml in ethanol. Plates were kept at 37° C. for 5 days to evaporate solvent completely. Cells were resuspended at 5×10⁴ cells/ml and were cultured (1 ml/well) in DMEM medium containing 10% fetal calf serum for different times on polyHEMA coated dishes at 37° C. and 5% CO₂. Subsequently, cells were subjected to cell viability studies using MTS or recovered and analyzed for caspase 3 activity.

ELISA for S100P

S100P was quantified in the media collected from S100P transfected NIH3T3 cells plated at 1×10⁵ cells per well for 3 days. S100P was captured between anti-S100 rabbit polyclonal antibody (Abcam Ltd., Cambridge, UK) and mouse monoclonal S100P antibody (Transduction Laboratories, San Diego, Calif.) and an ELISA kit (Protein Detector ELISA kit, KPL, Gaithersburg, Md., USA) following the manufactures recommendations. Anti-S100 Rabbit polyclonal antibody was coated in the ELISA plate and exposed to media from cells that had been concentrated by using YM10 centricon concentrating filter. Samples (200 μl) were incubated for 2 hrs at room temperature in antibody coated plates and washed thrice with wash buffer. Bound S100P was captured by using mouse monoclonal S100P antibody and subsequently with HRP labeled anti-mouse second antibody. TMB substrate was added and color development was blocked with 1M phosphoric acid and read at 450 nm. Purified S100P was used to plot graph as a standard and placental lysate was used as a positive control.

NF-κB Electrophoretic Mobility Shift Assay

Nuclear extracts were prepared and used for electrophoretic mobility shift assays (EMSAs) as previously described (Han et al., Am. J. Physiol. 277:C74 [1999]). For NF-κB DNA binding the reaction was started by addition of 10,000 cpm of the 22-base pair oligonucleotide 5′-AGT TGA GGG GAC TTT CCC AGG C-3′ (SEQ ID NO:7) containing the NF-κB consensus sequence that had been labeled with [γ-32P]-ATP (10 mCi/mmol) by T4 polynucleotide kinase. The reaction was allowed to proceed for 30 min at room temperature. For cold competition experiments unlabeled NF-κB oligonucleotide or OCT1 oligonucleotide as a nonspecific competitor (300×) were added to the binding reaction 5 min before the addition of the radiolabeled probe. For antibody supershift assays 2 μl of specific antibodies to NF-κB protein subunits p65, p50, and c-Rel were incubated with nuclear extracts for 1 hour at room temperature prior to the addition of labeled probe. All reaction mixtures were subjected to PAGE on 4.5% gel in 0.5×TBE buffer at 200 V. Gels were dried and directly exposed to the membranes were exposed to a B-1 phosphoimaging screen and visualized by the use of GS-250 Molecular Imaging System (Bio-Rad Laboratories, Richmond, Calif.).

B. Results

Expressed S100P Stimulates Cell Growth and Survival

Wild-type NIH3T3 cells do not express S100P. Therefore, to evaluate the influence of S100P on cell function NIH3T3 cells stably expressing this molecule were generated using standard transfection techniques (FIG. 4A). S100P expressing NIH3T3 cells were then analyzed for several cell functions. Initially, the effects of S100P on the ability of these cells to form colonies in soft-agar as an indication of cellular transformation was assessed. No significant colony formation was observed in NIH 3T3 cells either transiently or stably expressing S100P, suggesting a lack of transforming ability. However, S100P expression increased the proliferation rate of NIH3T3 by >200% of control cells within 96 h (FIG. 4B). This increase in proliferation rate correlated with an increase proportion of the cell population in S-phase (FIG. 4C).

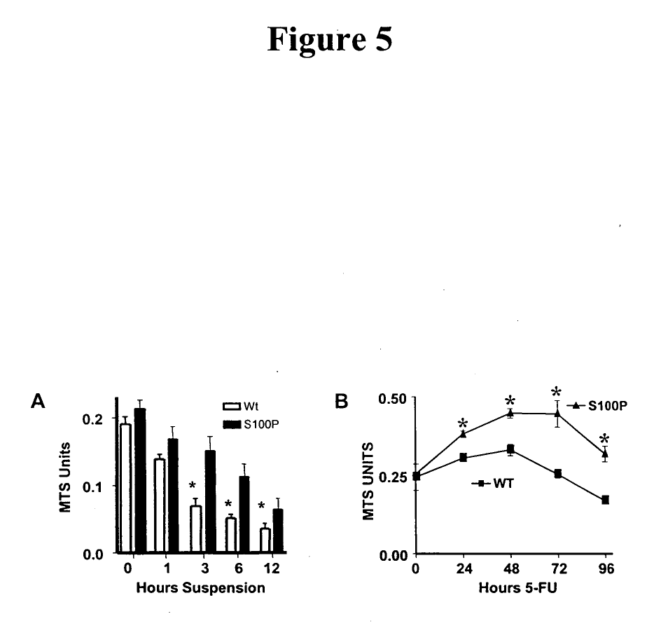

S100P expression also influenced NIH3T3 cell survival in the face of two apoptotic insults, removal from the growth substrate (causing anoikis) and treatment with the cytotoxic agent 5-FU. When wild-type NIH3T3 cells were plated on dishes coated with poly-Hema, which prevents cell attachment, the cells underwent rapid induction of anoikis indicated by a reduction in cell numbers (FIG. 5A). S100P expressing NIH3T3 cells were resistant to this treatment. The chemotherapeutic agent 5-FU was able to efficiently kill wild-type but not S100P expressing NIH3T3 cells (FIG. 5B). The survival benefits of S100P expression were due to an inhibition of apoptosis, as indicated by a reduced proportion of cells with sub-G1 levels of DNA content (FIG. 5C) and by a reduction in the appearance of active caspase 3 (FIG. 5C).

S100P is Secreted and Acts Extracellularly

Several S100 proteins have been found to act extracellularly to affect cell function. Therefore, the level of S100P in conditioned media from NIH3T3 cells stably transfected with S100P was examined. S100P was detectable in the medium bathing the cells using an ELISA assay levels (22 ng/ml, n=3).

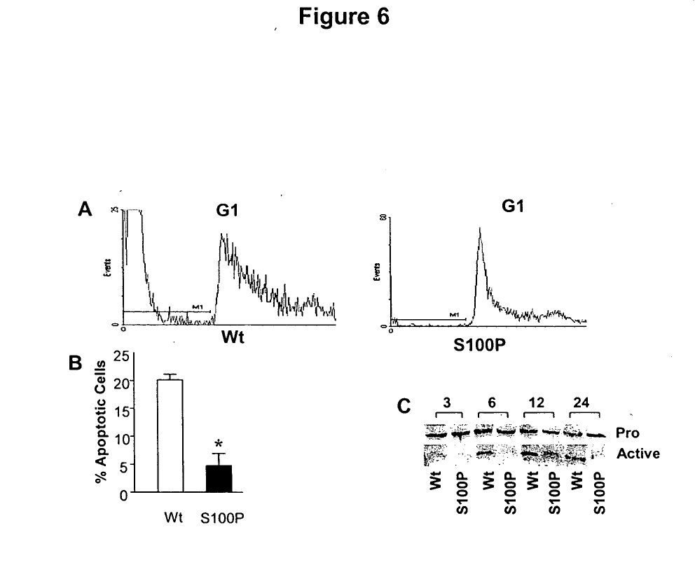

To test the effects of extracellular S100P on cell function, purified S100P was generated as a His-tagged fusion protein in bacteria (FIG. 6A). Addition of purified S100P to NIH3T3 cells stimulated cell proliferation in a dose-dependent manner. Effects were noted with 0.01 nM and maximal effects that were ˜2 fold over control were observed with 100 nM (FIG. 6B). The effects of S100P on cell proliferation were also time-dependent. A significant increase in cell proliferation was noted after 48 hours (FIG. 6C).

Addition of S100P to the culture medium also increased the survival of NIH3T3 cells after apoptotic insults. This protection was dose-dependent, with protection from the effects of 5 FU noted at 1 nM (FIG. 6D). The effects of S100P on cell survival were also time-dependent, with significant protection from anoikis noted after 36 hours (FIG. 6E).

S100P Activates Erk and NF-κB

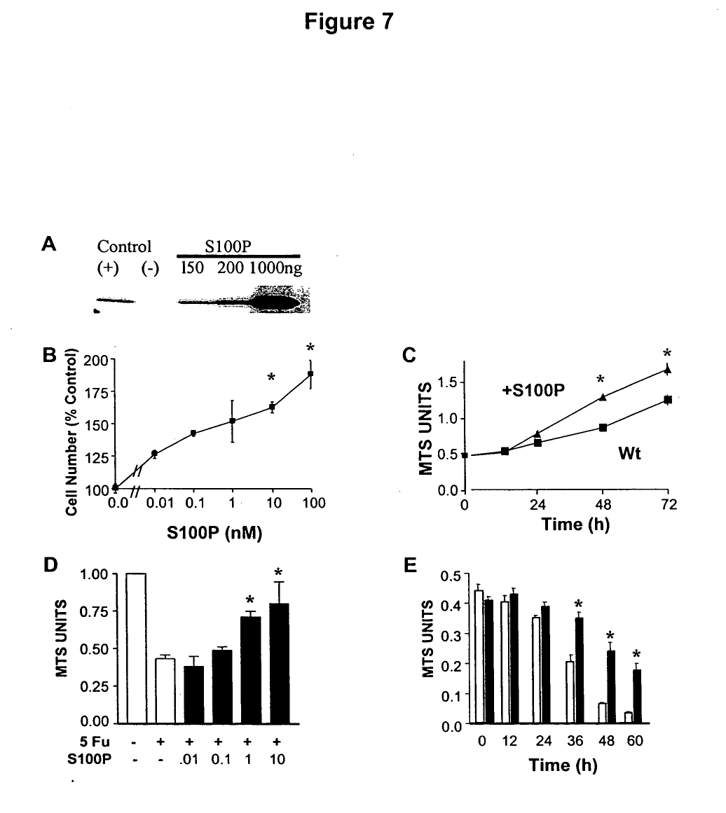

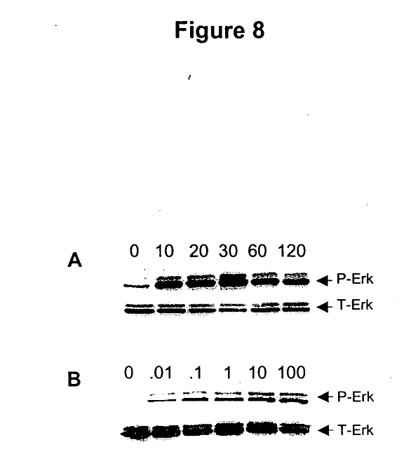

The effects of extracellular S100P on common cell growth and survival signaling pathways was next examined. Erk activation is commonly associated with stimulation of cell proliferation. Treatment of NIH3T3 cells with purified S100P induced Erk 1 & 2 phosphorylation in a time-dependent manner, with significant effects noted within 10 minutes and a maximal increase observed after 30 minutes (FIG. 7A). Beyond 30 minutes Erk phosphorylation levels returned towards base-line but remained significantly elevated for at least 2 hours. The effects of S100P on Erk phosphorylation were also dose-dependent, with effects noted at 0.01 nM and maximal effects noted with 100 nM (FIG. 7B).

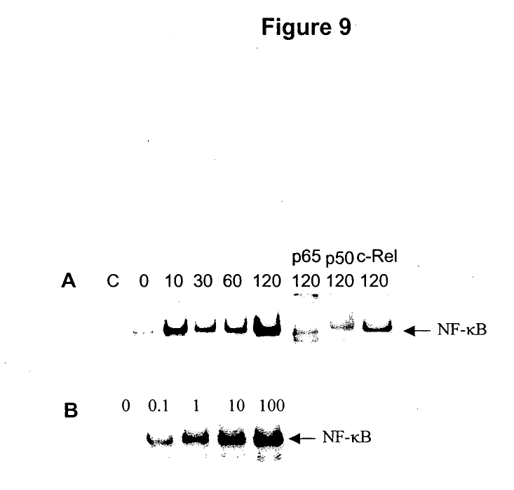

NF-κB activation is often associated with increase cell survival. Therefore, it was investigated whether extracellular S100P activates the transcription factor NFκB. S100P caused a time-dependent increase in NF-κB DNA binding in NIH3T3 cells that was initiated within 10 minutes and was maintained for at least 2 hours, as indicated by electrophoretic mobility shift assays (FIG. 9A). The specificity of the NF-κB band observed in these assays was indicated by competition with unlabeled κB site oligonucleotides. Furthermore, super-shift analysis using antibodies specific for individual NFκB subunits indicated the presence of p65, and p50 but not c-Rel in the induced complexes (FIG. 9A). These effects on NF-κB activation were also concentration dependent, with significant effects noted at 0.1 nM and a maximal effect observed with 100 nM S100P (FIG. 9B).

S100P Interacts with RAGE

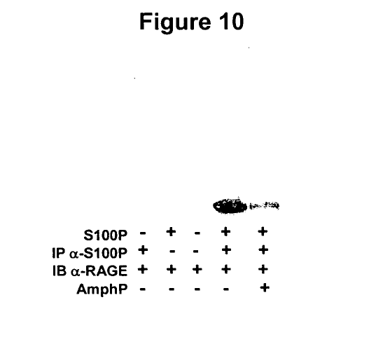

Previous studies revealed the interaction of several S100 molecules with RAGE. However, it is not known whether S100P can interact with RAGE. In order to investigate this possibility, pull-down assays were performed using lysates from NIH3T3 cells. Lysates were incubated with S100P and then S100P was immuno-precipitated and the isolated proteins were run on an SDS PAGE gel, transferred to nitrocellulose, and blotted with an antibody specific for RAGE. RAGE was not present in samples of wild-type NIH3T3 cells run without addition of S100P, or samples from wild-type or S100P expressing NIH3T3 cells without S100P antibody. However, RAGE was present in the S100P complexes from wild-type NIH3T3 cells incubated with exogenous S100P and S100P antibody (FIG. 10). Similarly, co-immuno-precipitation was observed with lysates from S100P expressing cells incubated with S100P antibody even in the absence of added S100P. These data indicate that S100P can interact directly with RAGE.

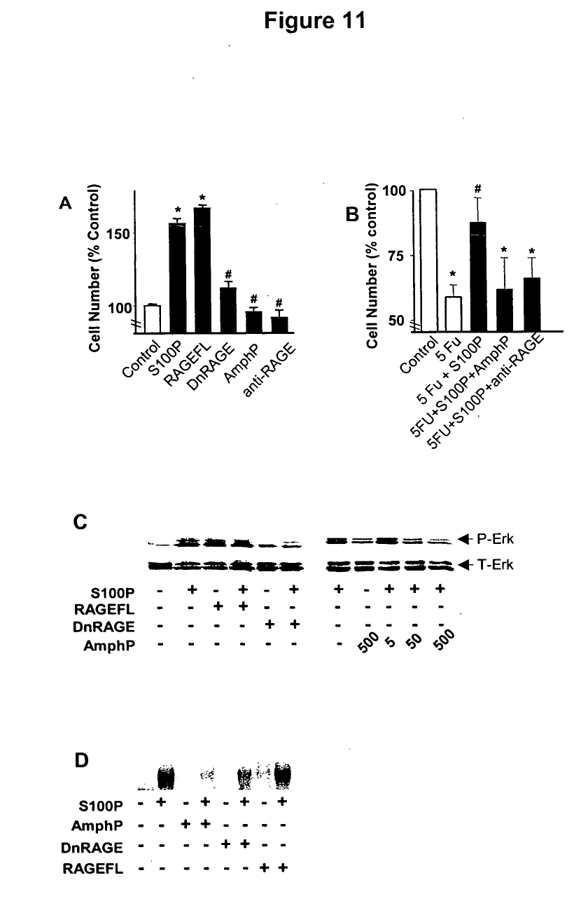

To determine whether or not S100P activation of RAGE was required for the effects of S100P on cell growth and survival, a variety of inhibitors were used to block the interaction of S100P with RAGE and the effects on cell function and signaling were investigated. Incubation with a synthetic peptide derived from amphoterin, a RAGE agonist, which has been previously shown to act as an antagonist for RAGE-amphoterin interactions (Huttuuunen et al., Cancer Res. 62:4805 [2002]) inhibited S100P RAGE interaction (FIG. 10). Incubation of wild-type NIH3T3 cells with this peptide, or with Fab2 fragments of anti-RAGE antibodies that have been previously shown to block RAGE activation (Taguchi et al., Nature 405:33354 [2000]) blocked the ability of S100P to stimulate cell growth (FIG. 11A) or protect cells from the effects of 5FU (FIG. 11B). Furthermore, overexpression of a truncated RAGE receptor that has previously been shown to act as a dominant negative (Taguchi et al., supra), but not a full-length RAGE receptor, blocked the ability of S100 to stimulate NIH3T3 cell growth (FIG. 11A) and to protect against the effects of 5FU (FIG. 11B). None of the inhibitors tested had any effects themselves on NIH3T3 cell function at the concentrations utilized.

Similar to their effects on cell proliferation and survival, inhibitors of S100P-RAGE interaction blocked the effects of S100P on NIH3T3 cell signaling. Thus, expression of DnRAGE but not full length RAGE inhibited S100P activation of Erks (FIG. 11C) and NF-κB (FIG. 11D). Likewise, the amphoterin peptide inhibited S100P activation of Erk (FIG. 11C) and NF-κB (FIG. 11D). The effects of the amphoterin peptide were concentration dependent with inhibitory effects on Erk activation noted at 50 nM and complete inhibition at 500 nM. Similar concentration dependence was observed for this peptide on S100P activation of NF-κB.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1. A method for characterizing pancreatic tissue in a subject, comprising:
a) providing a pancreatic tissue sample from a subject; and
b) detecting the presence or absence of expression of two or more genes selected from the group consisting of S100P, 14-3-3σ, β4 integrin, CEACAM5, PKM2, CST6, CST4, SERPINB5, FXYD3, BIK, SFN, TRIM29, ITGB4, NT5, IFI27 and S100A6.

2. The method of claim 1, wherein said detecting the presence of expression of two or more genes selected from the group consisting of S100P, 14-3-3σ, β4 integrin, CEACAM5, PKM2, CST6, CST4, SERPINB5, FXYD3, BIK, SFN, TRIM29, ITGB4, NT5, IFI27 and S100A6 comprises detecting the presence of mRNA expressed from said two or more genes.

3. The method of claim 2, wherein said detecting the presence of expression of mRNA expressed from said two or more genes comprises exposing said mRNA to a nucleic acid probe complementary to said mRNA.

4. The method of claim 1, wherein said detecting the presence of expression of two or more genes selected from the group consisting of S100P, 14-3-3σ, β4 integrin, CEACAM5, PKM2, CST6, CST4, SERPINB5, FXYD3, BIK, SFN, TRIM29, ITGB4, NT5, IFI27 and S100A6 comprises detecting the presence of a polypeptide expressed from said two or more genes.

5. The method of claim 4, wherein said detecting the presence of a polypeptide expression from said two or more genes comprises exposing said polypeptide to an antibody specific to said polypeptide and detecting the binding of said antibody to said polypeptide.

6. The method of claim 1, wherein said subject comprises a human subject.

7. The method of claim 1, wherein said sample comprises tumor tissue.

8. The method of claim 1, wherein said characterizing said pancreatic tissue comprises identifying a stage of pancreatic cancer in said pancreatic tissue.

9. The method of claim 1, further comprising the step of c) providing a prognosis to said subject.

10. The method of claim 9, wherein said prognosis comprises a risk of developing metastatic pancreatic cancer.

11. The method of claim 9, wherein said prognosis comprises a risk of developing pancreatic cancer.

12. The method of claim 1, further comprising the step of d) providing a diagnosis to said subject.

13. The method of claim 12, wherein said diagnosis comprises a diagnosis of pancreatic cancer.

14. The method of claim 13, wherein said diagnosis comprises a diagnosis of chronic pancreatitis.

15. A kit for characterizing pancreatic cancer in a subject, comprising:
a) a reagent capable of specifically detecting the presence of absence of expression of two or more genes selected from the group consisting of S100P, 14-3-3σ, β4 integrin, CEACAM5, PKM2, CST6, CST4, SERPINB5, FXYD3, BIK, SFN, TRIM29, ITGB4, NT5, IFI27 and S100A6; and
d) instructions for using said kit for characterizing cancer in said subject.

16. The kit of claim 15, wherein said reagent comprises a nucleic acid probe complementary to a mRNA expressed from said two or more genes.

17. The kit of claim 15, wherein said reagent comprises an antibody that specifically binds to a polypeptide encoded by said two or more genes.

18. The kit of claim 15, wherein said instructions comprise instructions required by the United States Food and Drug Administration for use in in vitro diagnostic products.

19. A method of screening compounds, comprising:
a) providing
i) a pancreatic cell sample; and
ii) one or more test compounds; and
b) contacting said pancreatic cell sample with said test compound; and
c) detecting a change in expression of two or more genes selected from the group consisting of S100P, 14-3-3σ, β4 integrin, CEACAM5, PKM2, CST6, CST4, SERPINB5, FXYD3, BIK, SFN, TRIM29, ITGB4, NT5, IFI27 and S100A6 in said pancreatic cell sample in the presence of said test compound relative to the absence of said test compound.

20. The method of claim 19, wherein said cell is selected from the group consisting of a cell in vitro and a cell in vivo.


SEQUENCE LISTING (7 sequences)

SEQ ID NO: 1 - DNA, 27 bp, Artificial Sequence
  Synthetic
atgacggaactagagacagccatgggc

SEQ ID NO: 2 - DNA, 27 bp, Artificial Sequence
  Synthetic
ggaatctgtgacatctccagcgcatca

SEQ ID NO: 3 - DNA, 26 bp, Artificial Sequence
  Synthetic
ccggatccctgtgtgtccccagagcc

SEQ ID NO: 4 - DNA, 26 bp, Artificial Sequence
  Synthetic
ccgaattcggcggtggcgggcaacac

SEQ ID NO: 5 - DNA, 23 bp, Artificial Sequence
  Synthetic
atgatatcgccgcgctcgtcgtc

SEQ ID NO: 6 - DNA, 20 bp, Artificial Sequence
  Synthetic
cgctcggccgtggtggtgaa

SEQ ID NO: 7 - DNA, 22 bp, Artificial Sequence
  Synthetic
agttgaggggactttcccaggc